(19) United States
(12) Patent Application Publication
(10) Pub. No.: US 2015/0157778 A1
(43) Pub. Date: Jun. 11, 2015

(54) MAGNETIC DRIVING PUMP DEVICE

(71) Applicants: Kazushi Ishiyama, Sendai-shi (JP); Syuji Matsumura, Saitama-shi (JP); Masaru Ozaki, Osaki-shi (JP)

(72) Inventors: Kazushi Ishiyama, Sendai-shi (JP); Syuji Matsumura, Saitama-shi (JP); Masaru Ozaki, Osaki-shi (JP)

(73) Assignees: TOHOKU UNIVERSITY, Miyagi (JP); I&P CO., LTD., Saitama (JP)

(21) Appl. No.: 14/390,815

(22) PCT Filed: Apr. 6, 2012
(86) PCT No.: PCT/JP2012/059548

§ 371 Date: Jan. 20, 2015

(51) Int. Cl. A61M 1/10

(52) U.S. Cl. CPC A61M 1/1036; A61M 1/1029; A61M 1/1086; A61M 2205/04; A61M 2205/103; A61M 2205/587; A61M 2205/3515; A61M 2205/3523; A61M 2205/33

(57) ABSTRACT

A magnetic driving pump device includes: an impeller; driven bodies integrally rotating together with the impeller and including permanent magnets; a drive unit including a magnet body on which a permanent magnet is placed such that when the drive unit rotates around a predetermined axis, different magnetic poles alternately appear on an outer periphery of the drive unit; and a rotation actuator rotating the magnet body around its axis. The magnet body is arranged side-by-side with respect to the driven bodies in a state that an axis of the magnet body is oriented to a direction which is not at right angles with respect to a rotation center of the impeller, and the impeller is rotated through the driven bodies by rotating the magnet body by the rotation actuator.

### FIELD

The present invention relates to a magnetic driving pump device including an impeller rotatably placed in a pump chamber of a pump case, and a drive unit provided outside the pump chamber, in which a rotating magnetic field is given from the drive unit to the impeller in a non-contact manner, thereby rotating the impeller with respect to the pump case, and more particularly, the invention relates to a magnetic driving pump device which is suitable as an artificial heart.

### BACKGROUND

As magnetic driving pump devices of this kind, a pump device described in Patent Literature 1 is provided for example. The pump device described in Patent Literature 1 includes an impeller rotatably placed in a pump chamber of a pump case, and a disk placed outside the pump chamber, in which the impeller and the disk are opposed to each other in a state where a rotation center of the impeller matches with a center of the disk. Permanent magnets are placed on portions of the impeller and the disk which correspond to each other such that the permanent magnets are located in their circumferential directions around rotation centers of the impeller and the disk. A magnetic pole of the permanent magnet of the impeller and a magnetic pole of the permanent magnet of the disk are opposite from each other, and an attractive force acts between the permanent magnets.

According to this magnetic driving pump device, if the disk is rotated by a rotation actuator, a rotating magnetic field can be given to the impeller placed in the pump chamber of the pump case in a non-contact manner, and it is possible to rotate the impeller with respect to the pump case.

According to the magnetic driving pump device of this kind, even in a state where the impeller and the disk must be in non-contact with each other, it is possible to maintain a state where the permanent magnets respectively placed on the impeller and the disk are always opposed to each other. Therefore, this pump device has many merits that power can efficiently be transmitted between the impeller and the disk and relatively large torque can be transmitted.

### CITATION LIST

### Patent Literature

Patent Literature 1: Japanese Laid-open Patent Publication No. 7-75667

### SUMMARY

### Technical Problem

It is desired that the magnetic driving pump device capable of rotating the impeller in a non-contact manner is applied to an artificial heart. That is, if a pump case in which an impeller is accommodated is implanted in a body and a disk connected to a rotation actuator is placed outside the body and the impeller can be rotated by rotating the disk from outside the body, it becomes unnecessary that a tube or a cable penetrates a skin. Therefore, burden of a patient can remarkably be reduced. Further, if the rotation actuator is configured such that it is driven by a battery, it is possible to broaden the scope of the patient's activity.

To rotate the impeller by the disk placed outside a body, the pump case must be implanted in the body in such a posture that the magnet of the impeller comes close to a body surface and a rotation center of the impeller is perpendicular to the body surface at right angles. Hence, a suction port of the pump case opens toward a deep portion of the body, a tube connected to the suction port extends toward the deep portion of the body and thus, an installation place of the pump case is largely limited. Further, to prevent hemolysis, it is difficult to increase the number of rotations of the impeller. Therefore, to increase a discharge amount, it is effective to coaxially place a plurality of impellers. However, a size of the pump device along an axial direction of the pump case is increased, and the installation place of the pump case is further limited.

If the pump case is provided with various kinds of sensors, it is possible to check, from outside a body, whether the impeller implanted in the body is normally operated. However, to drive the sensors, it is necessary to secure power. Hence, it is necessary that a cable penetrates a skin of a patient and burden of the patient is increased.

In view of the above circumstances, it is an object of the present invention to provide a magnetic driving pump device in which an installation place of the pump device is not largely limited and a pump case can be implanted. Further, it is an object of the invention to provide a magnetic driving pump device capable of easily detecting an operational state of an impeller from outside.

### Solution to Problem

To achieve the above-described object, a magnetic driving pump device according to the present invention includes: an impeller rotatably placed in a pump chamber of a pump case; driven bodies integrally rotating together with the impeller and including permanent magnets; and a drive unit provided outside the pump chamber, in which the impeller is rotated with respect to the pump case by giving a rotating magnetic field from the drive unit to the driven bodies in a non-contact manner, wherein the permanent magnets are placed on the driven bodies such that when the driven bodies are rotated, different magnetic poles alternately appear on outer peripheries of the driven bodies, the drive unit includes a magnet body on which a permanent magnet is placed such that when the drive unit rotates around a predetermined axis, different magnetic poles alternately appear on an outer periphery of the drive unit, and a rotation actuator rotating the magnet body around its axis, and the magnet body is arranged side-by-side with respect to the driven bodies in a state where an axis of the magnet body is oriented to a direction which is not at right angles with respect to a rotation center of the impeller, and the impeller is rotated through the driven bodies by rotating the magnet body by the rotation actuator.

Moreover, in the above-described magnetic driving pump device according to the present invention, each of the driven bodies is formed into a columnar shape whose axis corresponds to the rotation center of the impeller, and the permanent magnet of each of the driven bodies is placed on an outer peripheral surface of the driven body, and the magnet body of the drive unit is formed into a columnar shape whose axis corresponds to a rotation center of the magnet body, the permanent magnet of the drive unit is placed on an outer peripheral surface of the magnet body, and the magnet body of the drive unit is placed in a state where the outer peripheral surfaces of the magnet body of the drive unit and the driven bodies come close to each other.

Moreover, in the above-described magnetic driving pump device according to the present invention, the impeller is configured into a columnar outer shape such that an axis of the impeller corresponds to a rotation center of the impeller, respectively, the driven bodies are provided on both end surfaces of the impeller, and the magnet body of the drive unit has a length extending over peripheral surfaces of the driven bodies provided on the both end surfaces of the impeller.

Moreover, in the above-described magnetic driving pump device according to the present invention, outer diameters of the driven bodies match with an outer diameter of the impeller.

Moreover, in the above-described magnetic driving pump device according to the present invention, the impeller is integrally configured by the permanent magnets which configures the driven bodies.

Moreover, in the above-described magnetic driving pump device according to the present invention, an induction coil is placed in the pump case such that current flows in association with change in a magnetic field when the driven bodies rotate, and the pump case is provided with a control unit operated by current which flows through the induction coil.

Moreover, in the above-described magnetic driving pump device according to the present invention, an induction coil is placed in the pump case such that current flows in association with change in a magnetic field when the driven bodies rotate, and the pump case is provided with a light source which lights by current flowing through the induction coil.

Moreover, a magnetic driving pump device according to the present invention includes: an impeller rotatably placed in a pump chamber of a pump case; driven bodies which integrally rotate together with the impeller and which include permanent magnets; and a drive unit provided outside the pump chamber, in which the impeller is rotated with respect to the pump case by giving a rotating magnetic field from the drive unit to the driven bodies in a non-contact manner, wherein an induction coil is placed in the pump case such that current flows in association with change in a magnetic field when the driven bodies rotate, and the pump case is provided with a control unit operated by current which flows through the induction coil.

Moreover, a magnetic driving pump device according to the present invention includes: an impeller rotatably placed in a pump chamber of a pump case; driven bodies which integrally rotate together with the impeller and which include permanent magnets; and a drive unit provided outside the pump chamber, in which the impeller is rotated with respect to the pump case by giving a rotating magnetic field from the drive unit to the driven bodies in a non-contact manner, wherein an induction coil is placed in the pump case such that current flows in association with change in a magnetic field when the driven bodies rotate, and the pump case is provided with a light source which lights by current flowing through the induction coil.

Moreover, in the above-described magnetic driving pump device according to the present invention, the control unit includes a wireless communication device which detects an operational state of the impeller through a sensor, and which outputs a result of the detection to outside.

### Advantageous Effect of Invention

According to the present invention, the driven body which integrally rotates with the impeller and the magnet body rotated by the rotation actuator are arranged in directions which are not at right angles to each other, and the impeller is rotated by a driving operation of the rotation actuator. Therefore, the pump case can be implanted in such a posture that a rotation center of the impeller extends along a surface of the pump case, and there is no concern that the installation place of the pump case is largely limited. According to the present invention, the induction coil is placed in the pump case, and the light source and the control unit are operated by current which flows through the induction coil. Therefore, it is possible to detect an operational state of the impeller from outside without supplying power from outside.

### BRIEF DESCRIPTION OF DRAWINGS

FIG. 1 is a sectional side view of a magnetic driving pump device of an embodiment of the present invention.

FIG. 2 is a diagram illustrating an external appearance of the magnetic driving pump device illustrated in FIG. 1.

FIG. 3 is a sectional view schematically illustrating a state where a pump case of the magnetic driving pump device illustrated in FIG. 1 is placed in a body and a drive unit of the magnetic driving pump device is placed outside the body.

FIG. 4 is a perspective view of an external appearance of a rotor which is integrally formed together with an impeller and a driven body applied to the magnetic driving pump device illustrated in FIG. 1.

FIG. 5 is a block diagram for describing a function of the magnetic driving pump device illustrated in FIG. 1.

FIG. 6 is a diagram illustrating a layout of the magnetic driving pump device illustrated in FIG. 1.

### DESCRIPTION OF EMBODIMENT

A preferred embodiment of a magnetic driving pump device according to the present invention will be described below in detail with reference to the accompanying drawings.

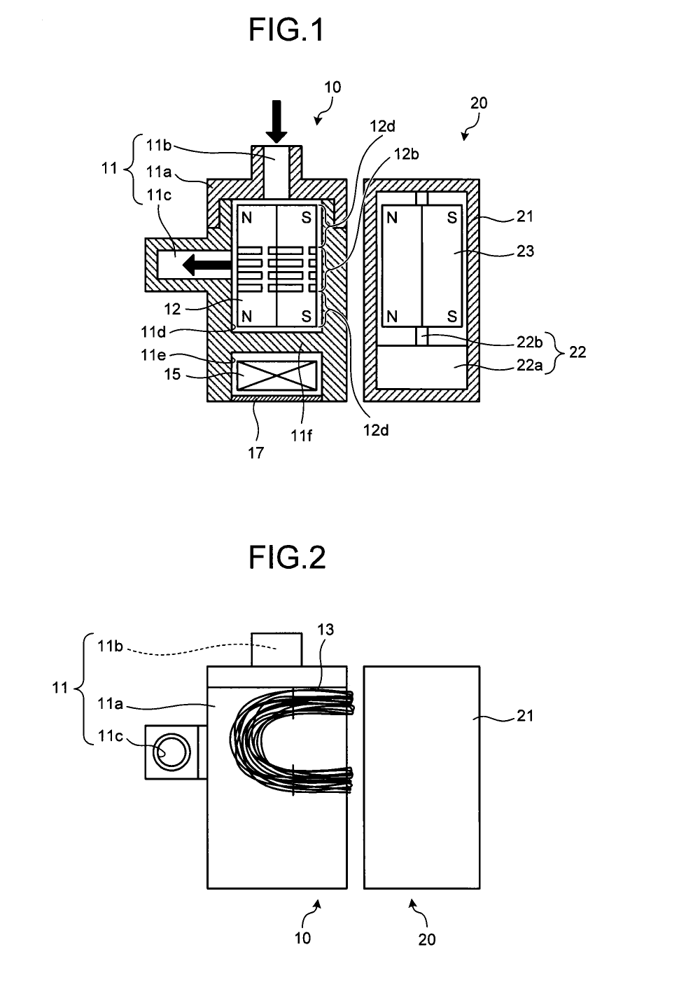

FIGS. 1 and 2 illustrate the magnetic driving pump device of the embodiment of the invention. The magnetic driving pump device shown here is configured on the premise that the pump device is applied as an artificial heart, and the pump device includes a pump unit 10 and a drive unit 20.

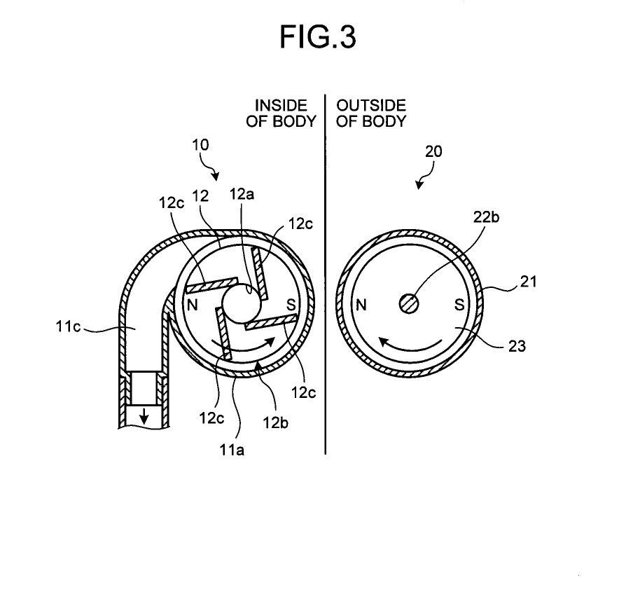

The pump unit 10 directly comes into contact with fluid and applies pressure, and includes a pump case 11. The pump case 11 includes a cylindrical case body 11a of which both ends are closed, a suction passage 11b provided in one of end surfaces of the case body 11a, and a discharge passage 11c provided in a peripheral surface of the case body 11a. These members are integrally formed of synthetic resin. The case body 11a has a palm-size and more particularly, an outer diameter thereof is about 20 mm and a length thereof is about 30 mm. The case body 11a is provided therein with a pump chamber 11d. The pump chamber 11d is a hollow having a circular transverse cross section, and the pump chamber 11d is in communication with outside through the suction passage 11b and the discharge passage 11c. A diameter of the suction passage 11b is smaller than that of the pump case 11, and the suction passage 11b is an extension of an axis of the pump chamber 11d. As illustrated in FIG. 3, the discharge passage 11c extends in a form of an arc along a circumferential direction from an outer peripheral surface of the pump case 11, and a base end of the discharge passage 11c opens into an inner peripheral surface of the pump chamber 11d.

As illustrated in FIGS. 1 and 3, a rotor 12 is placed in the pump chamber 11d in the pump case 11. The rotor 12 has an outer diameter slightly smaller than an inner diameter of the pump chamber 11d, the rotor 12 is formed into a columnar shape having a length slightly shorter than that of the pump chamber 11d, and the rotor 12 can rotate in the pump chamber 11d around an axis of the pump chamber 11d.

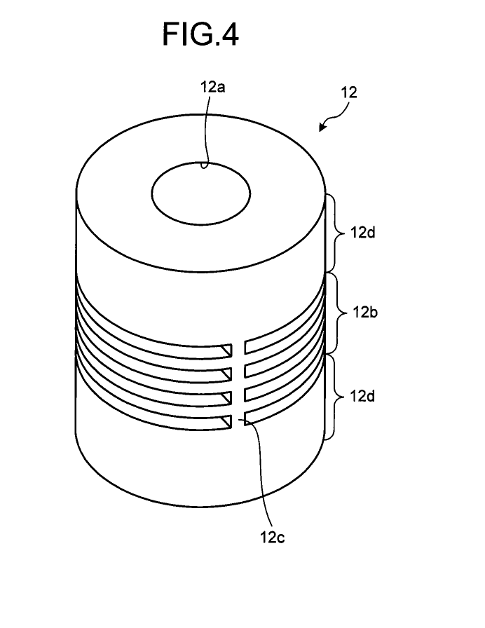

As illustrated in FIG. 4, a communication hole 12a is provided in a center portion of the rotor 12, and a plurality of impellers 12b are provided in an axially central portion of the rotor 12. The communication hole 12a is a through hole having an inner diameter substantially equal to an opening of the suction passage 11b, and the communication hole 12a has a circular transverse cross section. As illustrated in FIG. 1, the communication hole 12a is formed at position on an axis of the rotor 12. As illustrated in FIG. 3, a plurality of vanes 12c are placed in gaps of the impellers 12b which respectively open into an outer peripheral surface of the rotor 12 from the communication hole 12a, thereby radially configuring a plurality of flow paths. As illustrated in FIG. 1, four impellers 12b are arranged along the axial direction in a portion of the rotor 12 corresponding to an opening of the discharge passage 11c. In this embodiment, as illustrated in FIG. 3, four vanes 12c are placed at equal distances from one another along a radial direction from four peripheral locations of the communication hole 12a, and four flow paths whose widths are gradually increased toward an outer peripheral direction are provided. According to this, the impellers 12b are configured. Outer diameters of the impellers 12b are equal to each other, and the outer diameters of the impellers 12b match with an outer diameter of portions (“driven bodies 12d”, hereinafter) of the rotor 12 connected to both ends of the impellers 12b.

Although it is not clearly illustrated in the drawings, the rotor 12 is molded from plastic magnet material integrally with the impellers 12b and the driven bodies 12d by injection molding. Permanent magnets are placed in the rotor 12 over its entire length including its portions configuring the impellers 12b and the driven bodies 12d located on both ends of the rotor 12. More specifically, the permanent magnet is configured by polarizing the rotor 12 such that if the rotor 12 is divided into two pieces from a plane including its axis, one of the pieces becomes north pole over its entire length in the axial direction, and the other piece becomes south pole over its entire length in the axial direction. When the rotor 12 rotates around its axis, the north pole and the south pole alternately appear on the outer periphery of the rotor 12.

As illustrated in FIGS. 1 to 5, an induction coil 13, a light source 14 and a control unit 15 are placed in the pump case 11. The induction coil 13 is provided so that when the rotor 12 accommodated in the pump chamber 11d rotates, a magnetic field of the rotor 12 is changed and inductive current flows. The induction coil 13 is pasted on an outer peripheral surface of the pump case 11. A rectifier 16 is connected to the induction coil 13 for outputting induced alternating current as direct current. The light source 14 lights when current flows through the induction coil 13. A light-emitting diode is applied as the light source 14 in this embodiment. Although it is not clearly illustrated in the drawings, the control unit 15 is configured such that it has a desired function by mounting an electronic part on a circular circuit substrate, and the control unit 15 is placed in a unit accommodating portion 11e of the pump case 11 together with the rectifier 16 and the light source 14. The unit accommodating portion 11e is a recess provided in the other end surface of the pump case 11, and the unit accommodating portion 11e is closed with a lid member 17 in a state where the control unit 15 is accommodated. A partition wall 11f is provided between the unit accommodating portion 11e and the pump chamber 11d, and fluid does not flow between the unit accommodating portion 11e and the pump chamber 11d.

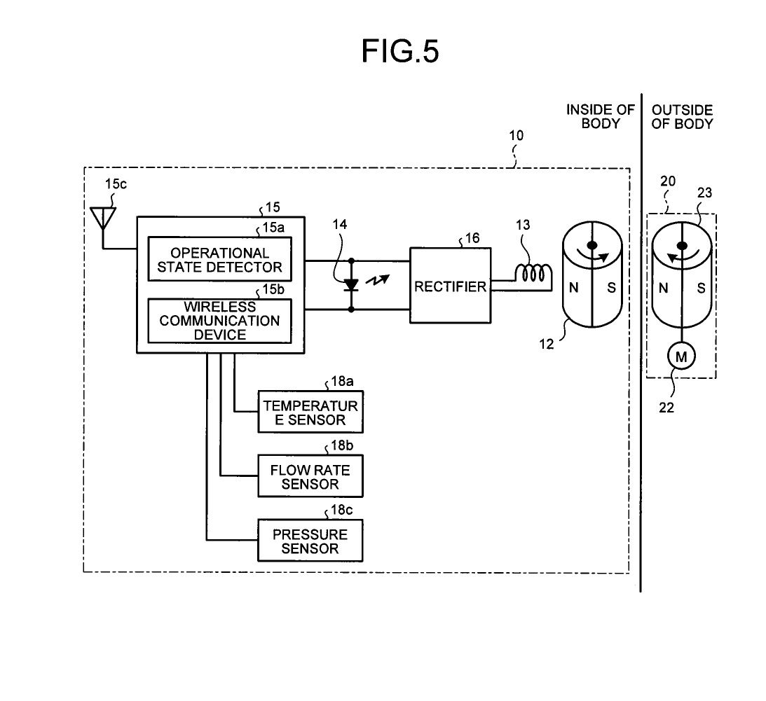

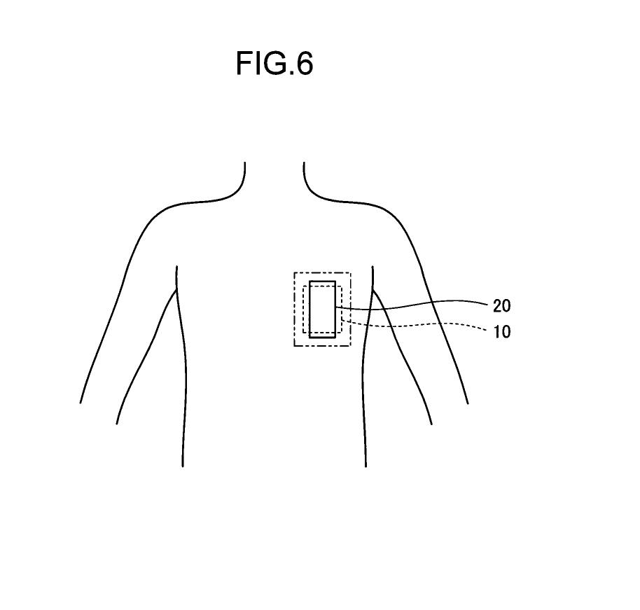

The control unit 15 is operated by current which flows through the induction coil 13. As illustrated in FIG. 5, the control unit 15 includes an operational state detector 15a and a wireless communication device 15b. The operational state detector 15a supplies electricity to various sensors 18a, 18b and 18c, and detects an operational state of the impellers 12b through detection signals which are output from these sensors 18a, 18b and 18c. In this embodiment, the temperature sensor 18a which detects temperature of the pump chamber 11d, the flow rate sensor 18b which detects a passing flow rate of the discharge passage 11c, and the pressure sensor 18c which detects pressure of fluid passing through the discharge passage 11c are provided at appropriate locations in the pump case 11. The operational state of the impellers 12b is detected through the detection signals of these sensors 18a, 18b and 18c. The wireless communication device 15b converts a detection result of the operational state detector 15a into transmitted data, and wirelessly sends the transmitted data to outside through an antenna 15c.

The drive unit 20 of the magnetic driving pump device is for giving, in a non-contact manner, a rotating magnetic field to the driven bodies 12d of the rotor 12 placed in the pump case 11, and the drive unit 20 includes a rotation actuator 22 provided in a drive unit case 21 which is configured separately from the pump case 11. The drive unit case 21 is cylindrical in shape, both ends of the drive unit case 21 are closed and the drive unit case 21 is formed of synthetic resin. The rotation actuator 22 is an electric motor having only one drive shaft 22b projecting from a body 22a of the rotation actuator 22. The rotation actuator 22 is fixed to an interior of the drive unit case 21 through the body 22a together with a battery (not illustrated) which is power.

A magnet body 23 is fixed to the drive shaft 22b of the rotation actuator 22. The magnet body 23 is formed into a columnar shape having an outer diameter which is substantially equal to a length of the rotor 12, and the magnet body 23 is placed in the drive unit case 21 in a state where the magnet body 23 can rotate around its own axis. Like the rotor 12, a permanent magnet is placed in a portion of the magnet body 23 over its entire length. More specifically, the permanent magnet is configured by polarizing the magnet body 23 such that if the magnet body 23 is divided into two pieces from a plane including its axis, one of the pieces becomes north pole over its entire length in the axial direction, and the other piece becomes south pole over its entire length in the axial direction. When the magnet body 23 rotates around its axis, the north pole and the south pole alternately appear on the outer periphery of the magnet body 23.

In the magnetic driving pump device configured as described above, the magnet body 23 and the driven bodies 12d are arranged side-by-side in a state where an axis of the magnet body 23 is oriented in a in direction which is not at right angles with respect to a rotation center of the impeller 12b, more preferably in a state where the axis of the magnet body 23 and the rotation center of the impeller 12b are parallel to each other and outer peripheral surfaces of the magnet body 23 and the impeller 12b are close to each other as illustrated in FIG. 1. At this time, as illustrated in FIG. 3 for example, if the magnet body 23 makes the north pole come close to the driven bodies 12d, the rotor 12 appropriately rotates in the pump case 11, and the driven bodies 12d make the south pole come close to the magnet body 23.

If the rotation actuator 22 of the drive unit 20 is driven from this state, magnetic poles which come close to the driven bodies 12d sequentially changes in the magnet body 23. In association with this, the driven bodies 12d, i.e., the impellers 12b rotate and follow this change in magnetic poles, and the pump unit 10 operates as a pump. Therefore, if tubes are respectively connected to the suction passage 11b and the discharge passage 11c of the pump case 11 and tip ends of the tubes are connected to blood vessels, blood sucked from the suction passage 11b can be discharged from the discharge passage 11c, and it is possible to make the magnetic driving pump device function as an artificial heart. Especially since the four impellers 12b are arranged side-by-side in the rotor 12, even when the rotor 12 is rotated with relatively low rotation number, it is possible to supply a sufficient amount of blood, and this is suitable for preventing hemolysis.

Here, if the magnet body 23 of the drive unit 20 is arranged in a direction which is not at right angles with respect to the rotation center of the impeller 12b, it is possible to give a rotating magnetic field from the magnet body 23 to the impellers 12b in a non-contact manner. According to this magnetic driving pump device, it is possible to implant the pump case 11 in a body in such a posture that the rotation center of the impeller 12b extends along a body surface. Therefore, there is no concern that the installation place of the pump case 11 is largely limited. In addition, as compared with a case where the pump case 11 is implanted toward a deep portion of a body, burden of a patient is remarkably reduced. Further, since the rotation actuator 22 is driven by a battery (not illustrated), a patient is not restrained by a cable or a tube. As illustrated in FIG. 6 for example, the drive unit 20 can be put in an inside pocket of clothes, and it is possible to broaden a scope of a patient's activity.

Since the induction coil 13 is placed in the pump case 11 and the light source 14 and the control unit 15 are operated by current which flows through the induction coil 13, it is possible to detect an operational state of the impellers 12b without supplying power from outside. That is, if the impellers 12b rotate and current flows through the induction coil 13, the light-emitting diode which is the light source 14 lights. Therefore, it is possible to determine whether the impellers 12b are rotating by checking this lighting state from outside a body. Further, the control unit 15 is operated by current which flows through the induction coil 13, a detection result of the temperature sensor 18a, a detection result of the flow rate sensor 18b and a detection result of the pressure sensor 18c are sent by the wireless communication device 15b, it is possible to monitor temperature in the pump chamber 11d, a flow rate of blood which passing through the discharge passage 11c, and pressure of blood which passes through the discharge passage 11c, and it is possible to detect the operational state of the impellers 12b in more detail.

Although the magnetic driving pump device is applied as the artificial heart in this embodiment, a use of the magnetic driving pump device is not necessarily limited to the artificial heart, and the magnetic driving pump device can also be used for other purposes.

Since the impellers 12b and the driven bodies 12d are integrally formed using plastic magnet material in the above-described embodiment, it is possible to make an attractive force act between the magnet body 23 of the drive unit 20, the impellers 12b and the driven bodies 12d, but it is unnecessary to integrally form the impellers 12b and the driven bodies 12d, and it is also unnecessary to configure the impellers 12b as permanent magnets.

Although the driven bodies 12d and the magnet body 23 are formed into the columnar shapes in the above-described embodiment, it is not absolutely necessary to form them into the columnar shapes, and it is only necessary that when the driven bodies 12d and the magnet body 23 rotate, the permanent magnets are placed such that different magnetic poles alternately appear on outer peripheries thereof.

### REFERENCE SIGNS LIST

11 pump case
11d pump chamber
12b impeller
12d driven body
13 induction coil
14 light source
15 control unit
15b wireless communication device
18a, 18b, 18c sensor
20 drive unit
21 drive unit case
22 rotation actuator
23 magnet body

1: A magnetic driving pump device comprising:
an impeller rotatably placed in a pump chamber of a pump case;
driven bodies integrally rotating together with the impeller and including permanent magnets placed on the driven bodies such that when the driven bodies are rotated, different magnetic poles alternately appear on outer peripheries of the driven bodies;
a drive unit provided outside the pump chamber and giving a rotating magnetic field to the driven bodies in a non-contact manner to rotate the impeller with respect to the pump case,
the drive unit including:
a magnet body on which a permanent magnet is placed such that when the drive unit rotates around a predetermined axis, different magnetic poles alternately appear on an outer periphery of the drive unit; and
a rotation actuator rotating the magnet body around its axis, wherein
the magnet body is arranged side-by-side with respect to the driven bodies in a state where an axis of the magnet body is oriented to a direction which is not at right angles with respect to a rotation center of the impeller, and the impeller is rotated through the driven bodies by rotating the magnet body by the rotation actuator.

2: The magnetic driving pump device according to claim 1, wherein
each of the driven bodies is formed into a columnar shape whose axis corresponds to the rotation center of the impeller, and the permanent magnet of each of the driven bodies is placed on an outer peripheral surface of the driven body, and
the magnet body of the drive unit is formed into a columnar shape whose axis corresponds to a rotation center of the magnet body, the permanent magnet of the drive unit is placed on an outer peripheral surface of the magnet body, and the magnet body of the drive unit is placed in a state where the outer peripheral surfaces of the magnet body of the drive unit and the driven bodies come close to each other.

3: The magnetic driving pump device according to claim 2, wherein
the impeller is configured into a columnar outer shape such that an axis of the impeller corresponds to a rotation center of the impeller, respectively, the driven bodies are provided on both end surfaces of the impeller, and the magnet body of the drive unit has a length extending over peripheral surfaces of the driven bodies provided on the both end surfaces of the impeller.

4: The magnetic driving pump device according to claim 3, wherein outer diameters of the driven bodies match with an outer diameter of the impeller.

5: The magnetic driving pump device according to claim 2, wherein the impeller is integrally configured by the permanent magnets which configures the driven bodies.

6: The magnetic driving pump device according to claim 1, further comprising an induction coil placed in the pump case such that current flows in association with change in a magnetic field when the driven bodies rotate, and the pump case is provided with a control unit operated by current which flows through the induction coil.

7: The magnetic driving pump device according to claim 1, further comprising an induction coil placed in the pump case such that current flows in association with change in a magnetic field when the driven bodies rotate, and the pump case is provided with a light source which lights by current flowing through the induction coil.

8. A magnetic driving pump device comprising:
an impeller rotatably placed in a pump chamber of a pump case;
driven bodies which integrally rotate together with the impeller and which include permanent magnets;
a drive unit provided outside the pump chamber, in which the impeller is rotated with respect to the pump case by giving a rotating magnetic field from the drive unit to the driven bodies in a non-contact manner; and
an induction coil placed in the pump case such that current flows in association with change in a magnetic field when the driven bodies rotate, and the pump case is provided with a control unit operated by current which flows through the induction coil.

9. A magnetic driving pump device comprising:
an impeller rotatably placed in a pump chamber of a pump case;
driven bodies which integrally rotate together with the impeller and which include permanent magnets;
a drive unit provided outside the pump chamber, in which the impeller is rotated with respect to the pump case by giving a rotating magnetic field from the drive unit to the driven bodies in a non-contact manner; and
an induction coil is placed in the pump case such that current flows in association with change in a magnetic field when the driven bodies rotate, and the pump case is provided with a light source which lights by current flowing through the induction coil.

10. The magnetic driving pump device according to claim 6, wherein the control unit includes a wireless communication device which detects an operational state of the impeller through a sensor, and which outputs a result of the detection to outside.

11. The magnetic driving pump device according to claim 8, wherein the control unit includes a wireless communication device which detects an operational state of the impeller through a sensor, and which outputs a result of the detection to outside.
